(19) United States
(12) Patent Application Publication
(10) Pub. No.: US 2024/0285508 A1
(43) Pub. Date: Aug. 29, 2024

(54) METHOD AND COMPOSITION FOR IMPROVING THE APPEARANCE OF SKIN

(71) Applicant: The Procter & Gamble Company, Cincinnati, OH (US)

(72) Inventors: Bin Fang Deyer, Loveland, OH (US); Tomohiro Hakozaki, Cincinnati, OH (US)

(21) Appl. No.: 18/443,597

(22) Filed: Feb. 16, 2024

(30) Foreign Application Priority Data
Feb. 16, 2023 (CA) 3190118

(51) Int. Cl. A61K 8/9783; A61K 8/60; A61Q 19/08

(52) U.S. Cl. CPC A61K 8/9783; A61K 8/602; A61Q 19/08

(57) ABSTRACT

A skin care composition that includes water lily extract, lactobionic acid, and a dermatologically acceptable carrier. The skin care composition can inhibit yellowness by greater than 50% and inhibits % glycation by greater than or equal to 35% according to the Advanced Glycation End Product Assay.

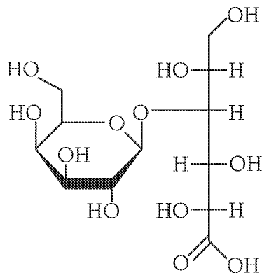

### FIELD

The present disclosure is directed generally to a cosmetic method of providing a skin health and/or appearance benefit and compositions therefor. More specifically, the present disclosure is directed to methods and compositions that utilize a combination of lactobionic acid and a Nymphaea alba (white water lily flower) extract to provide a skin benefit by inhibiting glycation and glycation associated yellowness formation.

### BACKGROUND

Skin is the first line of defense against environmental insults that would otherwise damage sensitive underlying tissue and organs. For example, skin maintains a relatively water-impermeable barrier between an organism and its environment to prevent dehydration. Additionally, skin plays a key role in a person's physical appearance. Generally, most people desire to have younger, healthy-looking skin. And to some of these people, the tell-tale signs of skin aging such as thinning skin, wrinkles, and age spots are an undesirable reminder of the disappearance of youth. As a result, treating the signs of aging in skin has become a booming business in youth-conscious societies. Treatments range from cosmetic creams and moisturizers to various forms of cosmetic surgery.

Glycation is one process that can make skin look older and less healthy. Glycation is generally recognized as a non-enzymatic process involving a reducing sugar that reacts with an amino group of an amino acid via a series of reactions characterized by the formation of advance glycation end products (“AGEs”). AGEs can lead to crosslinking of the proteins in skin, especially collagen and elastin, which can manifest as reduced skin elasticity, fine lines and wrinkles, and sallow looking skin. Glycation is also known to increase regularly with age and exposure to ultraviolet radiation.

Accordingly, it would be desirable to provide a cosmetic skin care composition that includes an anti-glycation active or a combination of such actives to prevent glycation in skin and sallow looking associated with glycation.

### SUMMARY

A skin care composition comprising: (a) a water lily extract comprising water lily plant material and/or a hydrolysate thereof; wherein the composition comprises from about 0.005% to about 0.05% of the water lily plant material and/or a hydrolysate thereof; (b) from about 0.05% to about 0.5% lactobionic acid; (c) a dermatologically acceptable carrier; wherein the composition inhibits % yellowness by greater than 50% and inhibits % glycation by greater than or equal to 35% according to the Advanced Glycation End Product Assay.

A skin care composition comprising: (a) a water lily extract comprising water lily plant material and/or a hydrolysate thereof; wherein the composition comprises from about 0.009% to about 0.015% of the water lily plant material and/or a hydrolysate thereof; (b) from about 0.1% to about 0.2% lactobionic acid; (c) a dermatologically acceptable carrier; wherein the composition inhibits % yellowness by greater than 53% and inhibits % glycation by greater than or equal to 35% according to the Advanced Glycation End Product Assay.

A skin care composition comprising: (a) about 0.4% Nymphae alba flower extract comprising Nymphae alba flower material; wherein the composition comprises from about 0.009% to about 0.015% of the Nymphae alba flower material and/or a hydrolysate thereof; (b) about 0.101% lactobionic acid; (c) a dermatologically acceptable carrier.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

The FIGURE represents the chemical structure of lactobionic acid.

### DETAILED DESCRIPTION

Numerous agents, both natural and synthetic, are known for use in skin care compositions marketed to treat various skin conditions, especially those associated with aging. One example of a well-known skin care agent is lactobionic acid (LBA). LBA is a polyhydroxy acid formed between beta-D-galactose and D-gluconic acid. It is used in the cosmetics industry to provide a variety of skin health benefits, including hydrating, antioxidant activity, inhibiting melanogenesis, and exfoliating.

Unfortunately, to deliver the desired benefit, LBA is generally used at high levels (e.g., ranging from 5-10%). Like all acids, LBA can irritate skin, especially dry or sensitive skin, and many current formulations are too strong.

Surprisingly, the Applicant discovered that a combination of LBA and Nymphaea alba (white water lily flower) extract in a topical skin care composition, in particular a composition with a relatively low level of lactobionic acid (e.g., from 0.1% to 0.2%) in combination with a specific level of Nymphaea alba extract (e.g., 0.4% white water lily extract), can deliver synergistic inhibition in vitro on both glycation and glycation associated sallowness/yellowness formation in the collagen matrix of skin according to the AGE Assay, described hereafter.

The composition can deliver a mean % Δb* (yellowness) inhibition, as determined by the Advanced Glycation End Product Assay (AGE Assay), described hereafter, ≥50%, alternatively ≥53%, alternatively ≥54%, alternatively ≥55%, alternatively ≥57%, alternatively ≥58%, alternatively ≥60%, and alternatively ≥70%.

The composition can deliver a mean % ΔFLR (glycation) inhibition, as determined by the AGE Assay, described hereafter, ≥35%, alternatively ≥38%, alternatively ≥40%, alternatively ≥ 41%, alternatively ≥43%, and alternatively ≥50%.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, less than 5%, or even less than 2%) of the stated value.

“Additive effect” means that the effect provided by a combination of actives is equal to or substantially equal to the sum of their individual effects. For example, an additive effect would be demonstrated when a first active would provide 10% improvement in the desired efficacy (i.e., inhibition of glycation or glycation associated yellowness), when being used alone, and a second active, which provides 15% improvement in the desired efficacy when used alone, provides a 25% improvement to the desired efficacy when used in combination. A “more than additive effect,” in this example, would be demonstrated when the combination of the first and second actives improves the desired efficacy by more than 25%.

“Apply” or “application”, as used in reference to a composition, means to apply or spread the compositions of the present invention onto a human skin surface such as the epidermis.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Cosmetic composition” means a composition comprising a cosmetic agent such as the water lily extract described herein. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein. In a specific example, effective amount means an amount of a water lily extract and LBA, in combination, sufficient to provide a desired benefit (e.g., synergistic inhibition in vitro on both glycation and glycation associated sallowness/yellowness formation in the collagen matrix of skin).

“Improve the appearance of” means providing a measurable, desirable change or benefit in a person's skin appearance, which may be quantified, for example, by a reduction in the Spot Arca Fraction of a hyperpigmented spot and/or a decrease in b* value of sallow skin. Exemplary methods for determining improvements in appearance are described in more detail below.

“L*a*b*” refers to the commonly recognized color space specified by the International Commission on Illumination (“CIE”). The three coordinates represent (i) the lightness of the color (i.e., L*=0 yields black and L*=100 indicates diffuse white), (ii) the position of the color between magenta and green (i.e., negative a* values indicate green while positive a* values indicate magenta) and (iii) the position of the color between yellow and blue (i.e., negative b* values indicate blue and positive b* values indicate yellow).

“Safe and effective amount” means an effective amount of an ingredient that is low enough to avoid serious side effects (within the scope of sound medical judgment).

“Sallow,” when referring to the appearance of skin herein, means an unusual yellow or pale skin tone, with regard to a particular individual, which is commonly associated with an unhealthy state. Sallow-appearing skin can be diagnosed objectively (e.g., with a color value such as L* or b*) or subjectively (e.g., by a skin care professional or consumer).

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Skin tone” means the overall appearance of basal skin color or color evenness. Skin tone is typically characterized over a larger area of the skin, which is generally more than 100 mm2, up to and including the entirety of the facial skin or other bodily skin surface (e.g., arms, legs, back, hands, neck, chest and abdomen). Skin tone can be measured by image analysis. One measure of skin tone is lightness, which can be measured by the L* coordinate in the L*a*b* color space (International Commission on Illumination). Chromophore mapping such as melanin mapping and melanin concentration may also be used as an indicator of skin tone. Mean melanin may be calculated from the chromophore map data. Additionally, skin tone can be correlated to melanin evenness (e.g., standard deviation) which also may be calculated from the chromophore map data.

“Synergy” and variations thereof mean that the effect provided by a combination of two or more materials (e.g., LBA and water lily extract) is more than the additive effect expected for these materials. For example, synergy is demonstrated when the white water lily extract in combination with LBA provide the desired protein glycation inhibition and associated yellowness inhibition according to Advanced Glycation End Product Assay.

“Treatment period,” as used herein, means the length of time and/or frequency that a material or composition is applied to a target skin surface.

“Vehicle control” means a negative control that is identical to the test composition except that it does not include the glycating reagent (e.g., DL-Glyceraldehyde) and testing material of interest (e.g., does not contain either lactobionic acid or water lily flower extract).

### Cosmetic Compositions

The compositions herein contain an effective amount of a combination of water lily extract obtained from a plant in the genus Nymphaea and LBA disposed in a dermatologically acceptable carrier and are intended for topical application to human skin. The amount of water lily extract and LBA should be sufficient to demonstrate synergistic inhibition in vitro on both glycation and glycation associated sallowness/yellowness formation in the collagen matrix of skin after a suitable course of treatment (e.g., 2, 4 or 8 weeks).

### Water Lily Extract

The compositions herein include an effective amount of water lily extract obtained from a plant in the genus Nymphaea. The water lily extract may be provided as a liquid (e.g., an aqueous solution containing water lily plant material and/or a hydrolysate thereof) or as a solid (e.g., a powder formed by drying the liquid water lily extract and/or a maltodextrin carrier thereof). The water lily extract may be mixed with a suitable carrier prior to incorporation into the cosmetic composition.

For example, powdered water lily extract may be mixed with a maltodextrin carrier (e.g., at a ratio of 1:9) or an aqueous carrier (e.g., water and/or a water soluble/miscible material). The amount of water lily extract that is effective can differ from one particular source of extract to another (i.e., supplier variation). However, the effective amount can be determined by the skilled artisan, for example, by measuring the level of protein glycation inhibition and associated yellowness inhibition according to Advanced Glycation End Product Assay, described hereafter. As with any extract, the concentration of active components and/or level of activity will depend on factors such as the final dilution volume of the extract product, the particular extraction method employed, the natural range of variation among individual plants, and other common factors known to those skilled in the art.

The water lily extract of the present invention may be obtained from a suitable species of water lily and/or portion of the water lily plant (e.g., flower, root, leaf, stem, seed, juice, and combinations of these), as long as the extract provides the desired protein glycation inhibition and associated yellowness inhibition according to Advanced Glycation End Product Assay. The water lily extract can be from Nymphaea alba flower.

The water lily extract may be in the form of a yellow, odorless, aqueous solution with a dry matter content of between about 10 and 55 g/L (e.g., between 15 and 45 g/L, between 20 and 30 g/L or even between 24 and 35). The active ingredient(s) obtained from water lily flower extract may contain a carbohydrate, protein or mineral, a hydrolysate of one or more of these or combination thereof. In some instances, the hydrolysate may include an enzymatic hydrolysate, obtained by conducting one or more enzymatic hydrolyses on the water lily extract or components thereof (e.g., carbohydrate or protein). When two more enzymatic hydrolyses are conducted, different types of enzymes may be used (e.g., a carbohydrase and a protease, two different carbohydrases, or two different proteases). The water lily extract may have a carbohydrate fraction of between 20% and 70% (e.g., 23% to 62% or 25% to 55%) by weight, based on the weight of the dry matter. The carbohydrate content can be determined according to the DUBOIS method 5 (Dubois M et al., Analytical Chemistry, 28, 3, 350-356, 1956) and expressed as a percentage of the total dry matter content of the water lily extract. In some instances, the water lily extract may have a mineral fraction of between 20% and 60% (e.g., 25% to 50% or even 30% to 45%) by weight, based on the weight of the dry matter. Mineral fraction can by determined by weighing the crude ash residue resulting from incineration of the samples of the active ingredient at 550° C. in an electric muffle furnace.

It may be desirable to minimize or eliminate the amount of peptides/proteins and/or polyphenols present in the water lily extract, which can reduce the risk of an allergic response or other adverse reaction from a user. Accordingly, the water lily extracts herein may have a peptide fraction of less than 20% (e.g., less than 15%, 10%, or even less than 5%) by weight, based on the weight of the dry matter, and may be free of or substantially free of polyphenol compounds (i.e., less than 3%, 1%, 0.5%, or even less than 0.2% by weight, based on the weight of the dry matter). The protein fraction of the water lily extract can be determined by the LOWRY method (Lowry et al., Protein measurement with the Folin reagent, J. Biol. Chem., 193, 265, 1951). In some instances, it may also be desirable to determine the size of the peptide compounds that are present, e.g., via FPLC type chromatography. The content of polyphenolic compounds can be determined according to the method provided in more detail below. The peptide/protein and/or polyphenol levels in the water lily extract may be provided as part of the initial process to obtain the water lily extract from the plant material or by subjecting the water lily extract to further processing.

In some instances, the water lily extract may be mixed with other suitable materials (e.g., water, thickeners, humectants, solvents, preservatives, and/or solubilizers) prior to incorporation into a composition. For example, the water lily extract may be mixed with water or another suitable carrier (e.g., a polyhydric alcohol such as glycerin) to provide a mixture containing 0.001% to 50% (e.g., 0.01% to 40%, 0.1% to 20%, 0.5% to 10%, or even 1% to 5%) water lily extract. The water lily extract and/or water lily mixture may be included in a composition at an amount of 0.00001% to 15%, 0.0001% to 10%, 0.001% to 15%, 0.025% to 10%, 0.05% to 10%, 0.05% to 5%, or even 0.1% to 5%, by weight. Water lily extract such as the one described above may be included in a composition at an amount of about 0.1% v/v to about 1% v/v, alternatively from about 0.2% v/v to about 0.7% v/v, and alternatively from about 0.3% v/v to about 0.5% v/v. In some examples the composition contains about 0.4% v/v water lily extract.

The skin care composition can contain from about 0.001% to about 0.1% water lily plant material and/or a hydrolysate thereof, alternatively from about 0.005% to about 0.05%, alternatively from about 0.007% to about 0.03%, alternatively from about 0.008% to about 0.04%, alternatively from about 0.008% to about 0.02%, alternatively 0.009% to about 0.015%, and alternatively from about 0.009% to about 0.014%. In some examples, the composition contains about 0.01% water lily plant material and/or a hydrolysate thereof.

### Lactobionic Acid (LBA)

The compositions herein include an effective amount of lactobionic acid. LBA can be identified as CAS No. 96-82-2 with the chemical formula C₁₂H₂₂O₁₂ and the structure in the FIGURE. An example of a commercially available lactobionic acid that may be suitable for use herein includes the TEGO® RenewHA Malto from Evonik®. The FIGURE represents the structure of LBA.

The composition can include from about 0.01% to about 0.2% LBA, alternatively from about 0.05% to about 0.2%, alternatively from about 0.07% to about 0.2%, alternatively about 0.08% to about 0.2%, and alternatively from about 0.1% to about 0.2% LBA.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil or oil-in-water). The oil phase of the present invention may comprise silicone oils, non-silicone oils such as hydrocarbon oils, esters, ethers, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). Emulsions may also contain an emulsifier, e.g., from about 1% to about 10% or from about 2% to about 5% based on the weight of the carrier. Emulsifiers may be nonionic, anionic or cationic. Some suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and Mccutcheon's Detergents and Emulsifiers, North American Edition, pages 317-324 (1986), each incorporated herein by reference.

The carrier may contain one or more dermatologically acceptable, hydrophilic diluents. As used herein, “diluent” includes materials in which the active ingredients, including the LBA and water lily extract, can be dispersed, dissolved, or otherwise incorporated. Hydrophilic diluents include water, organic hydrophilic diluents such as lower monovalent alcohols (e.g., C1-C4) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Vitamin B3 Compound

As used herein, “vitamin B3 compound” means a compound having the formula:

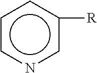

wherein R is —CONH₂ (i.e., niacinamide), —COOH (i.e., nicotinic acid) or —CH₂OH (i.e., nicotinyl alcohol); derivatives thereof; and salts of any of the foregoing. The vitamin B3 compound may be present at an amount of from about 0.00005% to about 15%, from about 0.0001% to about 10%, from about 0.001% to about 8%, from about 0.025% to about 7% from about 0.05% to about 7%, from about 0.5% to about 6%, from about 1% to about 6%, from about 2% to about 6%, or even from about 4% to about 6%, by weight of the total composition. In some examples, the composition can include about 5% of the vitamin B3 compound. A particular suitable vitamin B3 compound is niacinamide.

### Rheology Modifiers

The compositions herein may include 0.1% to 5% of a rheology modifier (e.g., thickening agent) to provide the composition with suitable rheological and skin feels properties. Some non-limiting examples of thickening agents include crosslinked polyacrylate polymers, polyacrylamide polymers, polysaccharides, gums and mixtures thereof. In a particularly suitable example, the composition may include a superabsorbent polymer thickening agent such as sodium polyacrylate, starch grafted sodium polyacrylate, or a combination of these. Some non-limiting examples of superabsorbent polymer thickeners are described in, for example, U.S. Pat. No. 9,795,552.

Some consumers find compositions that use silicone fluids as conditioning agents to be undesirably greasy or heavy feeling. Thus, it may be desirable to provide a composition that is free of or substantially free of silicone fluid. It may also be desirable to tailor a superabsorbent polymer thickener to provide the composition with a light, airy feel, for example, by adjusting the amount of water in the composition, the water:oil ratio (e.g., 12:1 to 1:1), and/or the ratio of water to thickener or oil to thickener.

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, anti-oxidants and/or antioxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition. In some instances, it may be desirable to select skin care actives that function via different biological pathways so that the actives do not interfere with one another, which could reduce the efficacy of both agents. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Product Form

The cosmetic compositions may be provided in various product forms that include, but are not limited to, solutions, suspensions, lotions, creams, gels, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. The cosmetic composition form may follow from the particular dermatologically acceptable carrier chosen, if present in the composition. The cosmetic compositions herein may be made using conventional methods of making such compositions.

### Method of Use

The present method includes identifying a target portion of skin (e.g., a facial skin surface such as the forehead, perioral, chin, periorbital, nose, and/or check) in need of treatment and applying a composition comprising a combination of specific level range of lactobionic acid and specific level of water lily extract and optionally one or more additional skin care agents, to the target portion of skin. In some instances, the target portion of skin may have a sallow skin tone or exhibit some other undesirable skin condition. In some instances, the target portion of skin may not appear to be suffering from a skin condition such as one associated with the effects of glycation, but a user (e.g., a relatively young user) may still wish to target such an area of skin if it is one that typically exhibits the undesirable effects of glycation or some other skin condition later in life (e.g., skin surfaces that are typically not covered by clothing, such as facial skin surfaces, hand and arm skin surfaces, foot and leg skin surfaces, and neck and chest skin surfaces). In this way, the present compositions may be used in a preventative capacity. The composition may be applied to the target skin portion and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. When used according to the methods herein, the present composition may improve the appearance of a skin condition by preventing glycation and glycation associated sallowness/yellowness.

The treatment period is ideally of sufficient time for a skin active present in the composition to improve the appearance of a target portion of skin. In some instances, the compositions may reduce glycation and sallowness/yellowness, for example, when compared to a vehicle control. The treatment period may last for at least 1 week (e.g., about 2 weeks, 4 weeks, 8 weeks, or even 12 weeks). In some instances, the treatment period will extend over multiple months (i.e., 3-12 months) or multiple years. In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., an area with visible glycation or portion thereof) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### Advanced Glycation End Product Assay (AGE Assay)

The AGE Assay provides an in vitro method of determining how a test agent impacts glycation. In particular, a material of interest can be applied to gelatin, which acts as a surrogate for collagen (i.e., a heterogeneous mixture of high-average-molecular-mass, water-soluble proteins present in collagen), at neutral pH range. A glycating reagent is added to the gelatin matrix to initialize glycation reaction. The resulting AGEs detected by a spectrophotometer. When testing a material of interest, such as lactobionic acid and/or white water lily extract is added to the gelatin sample, it reacts with the proteins in the gelatin, much in the same way as it would with collagen. Glycation reaction will be initiated when glycating reagent is added into this material and gelatin matrix and will produce AGEs. A positive (pos) glycation control group is set up the same way as testing material groups to contain gelatin and glycating reagent but less any treatment material. The positive control group depicts how much glycation and yellowness can be formed without perturbation by any treatment material. The spectrophotometer detects the fluorescence intensity (“FLR”) or yellowness (b*) associated with the resulting AGEs, which can then be correlated to the amount of glycation that occurs in skin. Lower b* and FLR values correspond to less AGEs, and thus less glycation.

Three replicates of each test samples are prepared in a 96-well plate (e.g., a FALCON brand 96-well tissue culture plate or equivalent) at equal and defined volume. The plate(s) containing the test samples are placed in a standard cell culture incubator (e.g., THERMO SCIENTIFIC FORMA brand incubator available from Fisher Scientific, Waltham, MA or equivalent) and incubated for the duration of the test at 37° C. with 5% CO₂ and 95% relative humidity. Fluorescence intensity (FLR) and yellowness are measured at the start of test (time=0) and monitored for 24 hours. To measure FLR or b*, the plate(s) containing the test samples are removed from the incubator and placed in a SPECTRAMAX Plus brand spectrophotometer (available from Molecular Devices, Sunnyvale, California) or equivalent. To detect fluorescence intensity (“FLR”), the spectrophotometer is set at 400/465 nm (ex/em). To detect yellowness (b*), the spectrophotometer is set to collect absorbance spectrum from 350 nm to 750 nm in 10 nm increments. The absorbance spectra from the yellowness measurement are then converted to L*a*b* values by a computer using suitable conversion software.

The change in fluorescence intensity (“ΔFLR”) is determined by comparing the FLR of a well at a sampling time point to the initial FLR at time=0. The change in yellowness (“Δb*”) at a particular time point is determined by comparing the initial b* value at time=0 to the b* value at the time point of interest. To determine the relative amount of glycation or yellowness formed due to a treatment effect compared to the positive glycation control group, which doesn't contain any active treatment, the ΔFLR and Δb* values from a treatment group is normalized against the corresponding values from the positive glycation control group (pos glycation) according to the following equation:

$$\% Glycation from a treatment relative to positive glycation control = \% Δ FLR = \frac{Δ {FLR}_{treatment}}{Δ FLRpos glycation} \times 100 \%$$

$$Therefore ,$$

$$Inhibition of glycation due to a treatment relative to positive glycation control group = 100 \% - \% Glycation from a treatment relative to positive glycation control$$

$$Detected \% Δ b * from a treatment relative to positive glycation control = \frac{Δ {b}_{treatment}^{*}}{Δ {b}_{pos glycation}^{*}} \times 100 \%$$

$$Therefore ,$$

$$Inhibition of yellowness due to a treatment relative to position glycation control group = 100 \% -$$

$$\% Y ellowness from a treatment relative to positive glycation control$$

### Examples

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

The effect on glycation and glycation associated yellowness was determined for the following testing materials:
lactobionic acid at 0.1%, 0.101%, and 0.2%.
white water lily extract (from Silab® containing 3.09% w/v dry matter of Nymphaea alba flower extract) at 0.4% v/v
a combination of both the lactobionic acid and the white water lily extract

The test samples were prepared in triplicate for each testing group on a 96-well plate with 250 μl volume per well. Each well contains 2.25 mg gelatin (available from Sigma® as Catalog #G1393), the testing material(s) diluted to desired concentration, 40 mM DL-Glyceraldehyde (available from Sigma®, Catalog #G5001-5G), and a sufficient quantity of Dulbecco's phosphate buffered saline (“DPBS”) (available from Gibco's, Catalog #14190-144). The vehicle control contained 2.25 mg gelatin and a sufficient quantity of DPBS. The positive glycation control contained 2.25 mg gelatin, 40 mM DL-glyceraldehyde and DPBS.

The average glycation, ΔFLR, and yellowness, Δb*, were determined for each sample according to the AGE Assay described herein. The duration of the test was 24 hours.

The data presented in Table 1 to Table 6, below, are normalized values against positive glycation control as described in the AGE Assay, described herein, to show relative % Glycation and relative % Yellowness as compared to positive glycation control group. In each case, inhibition of relative % Glycation and inhibition of relative % Yellowness was also calculated to demonstrate inhibiting effect as a result of treatment by a composition.

In Table 1 to Table 6, each table includes a row with a “calculated theoretical” combination. The results in this row are calculated, while the other results are detected. The % Δb* (yellowness) and % ΔFLR (glycation) are calculated by adding the impact of each component individually. If the tested combination is larger than the calculated theoretical combination, then the combination may demonstrate synergy.

Table 1 and Table 2, below, show the effect on % Yellowness inhibition and the % Glycation inhibition for 0.4% v/v white water lily extract and 0.1% and 0.2% LBA individually and 0.4% v/v white water lily extract in combination with 0.1% or 0.2% LBA. It was found that the % Yellowness inhibition and the Glycation inhibition by the combination of LBA and white water lily extract has more than an additive effect on inhibition, as compared to the ingredients separately, and therefore the combination is synergistic.

TABLE 1

| | % Δb* (yellowness) from | | | Convert to % Δb* | | | Mean |
|---|---|---|---|---|---|---|---|
| | a treatment vs positive | | | inhibition | | | % Δb* |
| | glycation control | | | (100%-% Δb*) | | | inhibition |
| | sample | sample | sample | sample | sample | sample | for each |
| Testing Material | 1 | 2 | 3 | 1 | 2 | 3 | group |
| 0.4% v/v white | 83 | 81 | 83 | 17 | 19 | 17 | 18 |
| water lily extract | | | | | | | |
| 0.1% LBA | 73 | 73 | 73 | 27 | 27 | 27 | 27 |
| Calculated | | | | 45 | 45 | 45 | 45 |
| theoretical 0.1% | | | | | | | |
| LBA + 0.4% v/v | | | | | | | |
| white water lily | | | | | | | |
| extract | | | | | | | |
| 0.1% LBA + 0.4% | 45 | 46 | 46 | 55 | 54 | 54 | 54 |
| v/v white water | | | | | | | |
| lily extract | | | | | | | |
| 0.2% lactobionic | 50 | 53 | 51 | 50 | 47 | 49 | 49 |
| acid | | | | | | | |
| Calculated | | | | 68 | 65 | 67 | 67 |
| theoretical 0.2% | | | | | | | |
| lactobionic acid + | | | | | | | |
| 0.4% v/v white | | | | | | | |
| water lily extract | | | | | | | |
| 0.2% lactobionic | 27 | 27 | 26 | 73 | 73 | 74 | 73 |
| acid + 0.4% white | | | | | | | |
| water lily mixture | | | | | | | |

TABLE 2

| | % ΔFLR (glycation) | | | Convert to % ΔFLR | | | Mean |
|---|---|---|---|---|---|---|---|
| | normalized vs positive | | | inhibition | | | % ΔFLR |
| | glycation control | | | (100-% ΔFLR) | | | inhibition |
| | sample | sample | sample | sample | sample | sample | for each |
| Testing Material | 1 | 2 | 3 | 1 | 2 | 3 | group |
| 0.4% white water | 95 | 94 | 94 | 5 | 6 | 6 | 6 |
| lily mixture | | | | | | | |
| 0.1% LBA | 77 | 77 | 75 | 23 | 23 | 25 | 24 |
| Calculated | | | | 29 | 29 | 30 | 29 |
| theoretical 0.1% | | | | | | | |
| LBA + 0.4% | | | | | | | |
| white water lily | | | | | | | |
| mixture | | | | | | | |
| 0.1% LBA + 0.4% | 59 | 59 | 60 | 41 | 41 | 40 | 41 |
| white water lily | | | | | | | |
| mixture | | | | | | | |
| 0.2% lactobionic | 55 | 56 | 56 | 45 | 44 | 44 | 44 |
| acid | | | | | | | |
| Calculated | | | | 50 | 49 | 50 | 50 |
| theoretical 0.2% | | | | | | | |
| lactobionic acid + | | | | | | | |
| 0.4% white | | | | | | | |
| water lily mixture | | | | | | | |
| 0.2% lactobionic | 36 | 37 | 37 | 64 | 63 | 63 | 63 |
| acid + 0.4% v/v | | | | | | | |
| white water lily | | | | | | | |
| extract | | | | | | | |

Table 3 and Table 4, below, show the effect on % Yellowness inhibition and the % Glycation inhibition for 0.4% v/v white water lily extract and 0.101% LBA individually and 0.4% v/v white water lily extract in combination with 0.101% LBA. It was found that the % Yellowness inhibition and the % Glycation inhibition by the combination of LBA and white water lily extract has more than an additive effect on inhibition, as compared to the ingredients separately, and therefore the combination is synergistic.

TABLE 3

| | % Δb* (yellowness) from | | | Convert to % Δb* | | | Mean |
|---|---|---|---|---|---|---|---|
| | a treatment vs positive | | | inhibition | | | % Δb* |
| | glycation control | | | (100-% Δb*) | | | inhibition |
| | sample | sample | sample | sample | sample | sample | for each |
| | 1 | 2 | 3 | 1 | 2 | 3 | group |
| 0.4% v/v white | 80 | 79 | 80 | 20 | 21 | 20 | 21 |
| water lily extract | | | | | | | |
| 0.101% lactobionic | 68 | 68 | 68 | 32 | 32 | 32 | 32 |
| acid | | | | | | | |
| Calculated 0.101% | | | | 53 | 53 | 52 | 53 |
| lactobionic acid + | | | | | | | |
| 0.4% v/v white | | | | | | | |
| water lily extract | | | | | | | |
| 0.101% lactobionic | | | 45 | 55 | 55 | 55 | 55 |
| acid + 0.4% v/v | | | | | | | |
| white water lily | 45 | 45 | | | | | |
| extract | | | | | | | |

TABLE 4

| | % ΔFLR (glycation) | | | Convert to % ΔFLR | | | Mean |
|---|---|---|---|---|---|---|---|
| | normalized vs positive | | | inhibition | | | % ΔFLR |
| | glycation control | | | (100-% ΔFLR) | | | inhibition |
| | sample | sample | sample | sample | sample | sample | for each |
| Testing Material | 1 | 2 | 3 | 1 | 2 | 3 | group |
| 0.4% v/v white | 94 | 93 | 93 | 6 | 7 | 7 | 7 |
| water lily extract | | | | | | | |
| 0.101% lactobionic | 73 | 74 | 73 | 27 | 26 | 27 | 27 |
| acid | | | | | | | |
| Calculated | | | | 34 | 33 | 34 | 34 |
| Theoretical 0.101% | | | | | | | |
| lactobionic acid + | | | | | | | |
| 0.4% v/v white | | | | | | | |
| water lily extract | | | | | | | |
| 0.101% lactobionic | 57 | 56 | 57 | 43 | 44 | 43 | 43 |
| acid + 0.4% v/v | | | | | | | |
| white water lily | | | | | | | |
| extract | | | | | | | |

Table 5 and Table 6, below, show the effect on % Yellowness inhibition and the % Glycation inhibition for 0.4% v/v white water lily extract and 0.1% LBA individually and 0.4% v/v white water lily extract in combination with 0.1% LBA. It was found that the % Yellowness inhibition and the % Glycation inhibition by the combination of LBA and white water lily extract has more than an additive effect on inhibition, as compared to the ingredients separately, and therefore the combination is synergistic.

TABLE 5

| | % Δb* (yellowness) from | | | Convert to % Δb* | | | Mean |
|---|---|---|---|---|---|---|---|
| | a treatment vs positive | | | inhibition | | | % Δb* |
| | glycation control | | | (100-% Δb*) | | | inhibition |
| | sample | sample | sample | sample | sample | sample | for each |
| Testing Material | 1 | 2 | 3 | 1 | 2 | 3 | group |
| 0.4% v/v white | 82 | 82 | 83 | 18 | 18 | 17 | 18 |
| water lily extract | | | | | | | |
| 0.1% LBA | 68 | 69 | 67 | 32 | 31 | 33 | 32 |
| Calculated | | | | 49 | 49 | 50 | 49 |
| Theoretical 0.1% | | | | | | | |
| LBA + 0.4% v/v | | | | | | | |
| white water lily | | | | | | | |
| extract | | | | | | | |
| 0.1% LBA + 0.4% | 42 | 42 | 42 | 58 | 58 | 58 | 58 |
| v/v white water | | | | | | | |
| lily extract | | | | | | | |

TABLE 6

| | % ΔFLR (glycation) | | | Convert to % ΔFLR | | | Mean |
|---|---|---|---|---|---|---|---|
| | normalized vs positive | | | inhibition | | | % ΔFLR |
| | glycation ctrl | | | (100-% ΔFLR) | | | inhibition |
| | sample | sample | sample | sample | sample | sample | for each |
| Testing Material | 1 | 2 | 3 | 1 | 2 | 3 | group |
| 0.4% v/v white | 94 | 94 | 95 | 6 | 6 | 5 | 6 |
| water lily extract | | | | | | | |
| 0.1% LBA | 69 | 71 | 70 | 31 | 29 | 30 | 30 |
| Calculated | | | | 37 | 35 | 35 | 36 |
| Theoretical 0.1% | | | | | | | |
| LBA + 0.4% v/v | | | | | | | |
| white water lily | | | | | | | |
| extract | | | | | | | |
| 0.1% LBA + 0.4% | 53 | 55 | 55 | 47 | 45 | 45 | 46 |
| v/v white water | | | | | | | |
| lily extract | | | | | | | |

Table 7 and Table 8, below, includes skin care compositions that could be made with a synergistic combination of water lily extract and LBA.

TABLE 7

| | Ex. A | Ex. B | Ex. C | Ex. D | Ex. E |
|---|---|---|---|---|---|
| Phase A | | | | | |
| Water | | | | | |
| Niacinamide | 1.000 | — | 3.500 | — | 2.000 |
| Dexpanthenol | 0.250 | 0.250 | 0.500 | 1.000 | |
| Nymphaea Alba Flower Extract¹ | 0.400 | 0.400 | 0.400 | 0.400 | 0.400 |
| Lactobionic acid | 0.101 | 0.101 | 0.101 | 0.101 | 0.101 |
| Glycerol | 5.000 | 7.000 | 3.000 | 15.000 | 7.000 |
| Disodium EDTA | 0.100 | 0.050 | 0.100 | 0.100 | 0.050 |
| Phase B | — | — | — | — | — |
| Dimethicone 5 cst | — | — | — | — | — |
| Dimethicone and Dimethiconol | 0.500 | 1.000 | 2.000 | 1.000 | 2.000 |
| Dimethicone (and) Dimethicone/ | — | — | — | — | — |
| Vinyl Dimethicone Crosspolymer | | | | | |
| Laureth 4 | — | — | — | — | — |
| Polysorbate 20 | — | — | — | — | — |
| Tapioca Starch and | — | — | — | — | — |
| Polymethylsilsesquioxane | | | | | |
| Lauroyl Lysine | 1.000 | 2.000 | 2.000 | — | — |
| Avobenzone | — | — | — | 3.000 | — |
| Homosalate | — | — | — | 15.000 | — |
| Octisalate | — | — | — | 5.000 | — |
| Octocrylene | — | — | — | 2.600 | — |
| Isopropyl Isostearate | 5.000 | 2.500 | 1.000 | — | — |
| Isohexadecane | 1.000 | 1.500 | 3.000 | — | — |
| Cetyl Alcohol | 0.250 | 0.500 | 0.320 | 0.400 | 0.400 |
| Tocopherol actetate | | 0.500 | 0.250 | 1.000 | 0.250 |
| PEG-100 Stearate | 0.200 | 0.100 | 0.100 | 0.300 | 0.100 |
| Stearyl Alcohol | 0.500 | 1.500 | 0.400 | 0.600 | 0.500 |
| Behenyl Alcohol | 0.400 | 1.000 | 0.500 | 0.500 | 0.400 |
| Ethyl Paraben | — | — | — | 0.200 | 0.250 |
| Propyl Paraben | — | — | — | 0.100 | 0.150 |
| Hydroxyacetophenone | 0.150 | 0.150 | 0.150 | — | — |
| Phase C | | | | | |
| Titanium Dioxide | — | 0.500 | | 0.250 | — |
| Sodium Polyacrylate Starch | | — | | | — |
| Hydroxyethyl acrylate/sodium | 2.000 | — | 2.500 | 2.000 | — |
| acryloyldimethyltaurate | | | | | |
| copolymer | | | | | |
| Polyacrylate Crosspolymer 6 | — | — | — | — | — |
| Sodium Polyacryloyldimethyl | — | — | — | — | 1.500 |
| Taurate | | | | | |
| Polyacrylamide & C13-14 | — | — | — | — | |
| Isoparaffin & Laureth-7 | | | | | |
| Sodium Acrylate/Sodium | — | 1.500 | — | — | — |
| Acryloyldimethyl Taurate | | | | | |
| Copolymer (and) Isohexadecane | | | | | |
| (and) Polysorbate 80 | | | | | |
| Xanthan Gum | — | 0.100 | — | — | — |
| Phase D | | | | | |
| Benzyl alcohol | 0.250 | 0.400 | 0.250 | 0.500 | — |
| Hexanediol and Caprylyl glycol | — | — | — | — | 0.700 |
| Phenoxyethanol | — | — | — | — | 0.300 |

TABLE 8

| | Ex. F | Ex. G | Ex. H | Ex. I |
|---|---|---|---|---|
| Phase A | | | | |
| Water | | | | |
| Niacinamide | 5.000 | — | — | 2.000 |
| Dexpanthenol | 0.500 | | 1.000 | 1.000 |
| Nymphaea Alba Flower Extract¹ | 0.400 | 0.400 | 0.400 | 0.400 |
| Lactobionic acid | 0.101 | 0.101 | 0.101 | 0.101 |
| Glycerol | 5.000 | 5.000 | 3.000 | 5.000 |
| Disodium EDTA | 0.050 | 0.050 | 0.050 | 0.100 |
| Phase B | — | — | — | — |
| Dimethicone 5 cst | — | — | 10.000 | 15.000 |
| Dimethicone and Dimethiconol | 2.000 | 1.000 | 1.750 | 1.000 |
| Dimethicone (and) Dimethicone/ | — | — | 13.000 | 15.000 |
| Vinyl Dimethicone Crosspolymer | | | | |
| Laureth 4 | — | — | 0.250 | 0.350 |
| Polysorbate 20 | — | — | 0.150 | 0.250 |
| Tapioca Starch and | — | — | 2.000 | 4.000 |
| Polymethylsilsesquioxane | | | | |
| Lauroyl Lysine | 4.000 | — | — | — |
| Avobenzone | 3.000 | — | — | — |
| Homosalate | 10.000 | — | — | — |
| Octisalate | 5.000 | — | — | — |
| Octocrylene | 9.000 | — | — | — |
| Isopropyl Isostearate | — | — | — | — |
| Isohexadecane | — | — | — | — |
| Cetyl Alcohol | 0.300 | 0.500 | — | — |
| Tocopherol actetate | 0.250 | 0.250 | — | — |
| PEG-100 Stearate | 0.200 | 0.100 | — | — |
| Stearyl Alcohol | 0.400 | 0.600 | — | — |
| Behenyl Alcohol | 0.350 | 0.500 | — | — |
| Ethyl Paraben | — | — | — | — |
| Propyl Paraben | — | — | — | — |
| Hydroxyacetophenone | — | — | 0.150 | 0.150 |
| Phase C | | | | |
| Titanium Dioxide | — | — | — | — |
| Sodium Polyacrylate Starch | 1.000 | — | — | — |
| Hydroxyethyl acrylate/sodium | — | — | | — |
| acryloyldimethyltaurate | | | | |
| copolymer | | | | |
| Polyacrylate Crosspolymer 6 | — | — | 1.200 | — |
| Sodium Polyacryloyldimethyl | — | — | — | 1.500 |
| Taurate | | | | |
| Polyacrylamide & C13-14 | | 2.000 | — | |
| Isoparaffin & Laureth-7 | | | | |
| Sodium Acrylate/Sodium | — | — | — | — |
| Acryloyldimethyl Taurate | | | | |
| Copolymer (and) Isohexadecane | | | | |
| (and) Polysorbate 80 | | | | |
| Xanthan Gum | — | — | — | — |
| Phase D | | | | |
| Benzyl alcohol | — | — | — | — |
| Hexanediol and Caprylyl glycol | 0.800 | 0.700 | | |
| Phenoxyethanol | 0.400 | 0.500 | 0.250 | 0.250 |

¹White water lily extract from Silab ® containing 3.09% w/v dry matter of Nymphaea alba flower extract

### Combinations:

A. A skin care composition comprising:
   a. a water lily extract comprising water lily plant material and/or a hydrolysate thereof;
      wherein the composition comprises from about 0.005% to about 0.05% of the water lily plant material and/or a hydrolysate thereof;
   b. from about 0.05% to about 0.5% lactobionic acid, preferably from about 0.1% to about 0.2% lactobionic acid;
   c. a dermatologically acceptable carrier;
      wherein the composition inhibits % yellowness by greater than 50%, preferably by greater than 53% according to the Advanced Glycation End Product Assay; and
      inhibits 9% glycation by greater than or equal to 35% according to the Advanced Glycation End Product Assay.
B. The skin care composition according to Paragraph A, wherein the composition comprises from about 0.008% to about 0.02% of the water lily plant material and/or the hydrolysate thereof, preferably from about 0.009% to about 0.015% of the water lily plant material and/or the hydrolysate thereof.
C. The skin care composition according to Paragraphs A-B, wherein the water lily plant material and/or the hydrolysate thereof is obtained from a flower of Nymphaea alba.
D. The skin care composition according to Paragraphs A-C, wherein plant material and/or a hydrolysate thereof comprises about 20% to about 70% carbohydrate based on the weight of the dry matter.
E. The skin care composition according to Paragraphs A-D, wherein the plant material and/or a hydrolysate thereof comprises about 20% to about 60% mineral based on the weight of the dry matter.
F. The skin care composition according to Paragraphs A-E, wherein the water lily plant material and/or a hydrolysate thereof comprises less than about 20% protein, based on the weight of the dry matter.
G. The skin care composition according to Paragraphs A-F, wherein the water lily plant material and/or a hydrolysate thereof comprises less than about 3% polyphenol based on the weight of the dry matter.
H. The skin care composition according to Paragraphs A-G, wherein the composition further comprises from about 1% to about 6% of a of vitamin B3 compound, preferably about 5% of a vitamin B3 compound.
I. The skin care composition according to Paragraph H, wherein the vitamin B3 compound comprises niacinamide.
J. The skin care composition according to Paragraphs A-I, wherein the composition comprises about 0.1% to about 0.15% lactobionic acid.
K. The skin care composition according to Paragraphs A-J, wherein the composition comprises about 0.1% lactobionic acid.
L. The skin care composition according to Paragraphs A-J, wherein the composition comprises about 0.101% lactobionic acid.
M. The skin care composition according to Paragraphs A-I, wherein the composition comprises about 0.2% lactobionic acid.
N. The skin care composition according to Paragraphs A-M, wherein the composition comprises about 0.4% of the water lily extract.
O. The skin care composition according to Paragraphs A-N, wherein the water lily extract is Nymphae alba flower extract comprising Nymphae alba flower material.
P. The skin care composition according to Paragraphs A-O, further comprising:
   a. silicone oils;
   b. glycerol;
   c. disodium EDTA;
   d. lauroyl lysine;
   e. xanthan gum;
   f. benzyl alcohol; and
   g. phenoxyethanol.
Q. A method of improving the appearance of skin, comprising:
   a. identifying a target portion of skin where treatment is desired; and
   b. applying a composition comprising the composition of Paragraphs A-P to the target portion of skin during a treatment period, wherein the composition inhibits % yellowness by greater than 50% and inhibits % glycation by greater than or equal to 35% according to the Advanced Glycation End Product Assay.
R. The method of Paragraph Q, wherein the target portion of skin exhibits a sign of glycation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A skin care composition comprising:
a. a water lily extract comprising water lily plant material and/or a hydrolysate thereof;
wherein the composition comprises from about 0.005% to about 0.05% of the water lily plant material and/or a hydrolysate thereof;
b. from about 0.05% to about 0.5% lactobionic acid;
c. a dermatologically acceptable carrier;
wherein the composition inhibits % yellowness by greater than 50% and inhibits % glycation by greater than or equal to 35% according to the Advanced Glycation End Product Assay.

2. The skin care composition of claim 1, wherein the composition comprises from about 0.008% to about 0.02% of the water lily plant material and/or the hydrolysate thereof.

3. The skin care composition of claim 2, wherein the composition comprises from about 0.009% to about 0.015% of the water lily plant material and/or the hydrolysate thereof.

4. The skin care composition of claim 1, wherein the water lily plant material and/or the hydrolysate thereof is obtained from a flower of Nymphaea alba.

5. The composition of claim 1, wherein plant material and/or a hydrolysate thereof comprises about 20% to about 70% carbohydrate based on the weight of the dry matter.

6. The composition of claim 1, wherein the plant material and/or a hydrolysate thereof comprises about 20% to about 60% mineral based on the weight of the dry matter.

7. The composition of claim 1, wherein the water lily plant material and/or a hydrolysate thereof comprises less than about 20% protein, based on the weight of the dry matter.

8. The composition of claim 1, wherein the water lily plant material and/or a hydrolysate thereof comprises less than about 3% polyphenol based on the weight of the dry matter.

9. The skin care composition of claim 1, wherein the composition further comprises from about 1% to about 6% of a of vitamin B3 compound.

10. A skin care composition comprising:
a. a water lily extract comprising water lily plant material and/or a hydrolysate thereof;
wherein the composition comprises from about 0.009% to about 0.015% of the water lily plant material and/or a hydrolysate thereof;
b. from about 0.1% to about 0.2% lactobionic acid;
c. a dermatologically acceptable carrier;
wherein the composition inhibits % yellowness by greater than 53% and inhibits % glycation by greater than or equal to 35% according to the Advanced Glycation End Product Assay.

11. The skin care composition of claim 10, wherein the composition comprises about 0.1% to about 0.15% lactobionic acid.

12. The skin care composition of claim 11, wherein the composition comprises about 0.1% lactobionic acid.

13. The skin care composition of claim 10, wherein the composition comprises about 0.2% lactobionic acid.

14. The skin care composition of claim 10, wherein the composition further comprises about 5% of a of vitamin B3 compound.

15. A skin care composition comprising:
a. about 0.4% Nymphae alba flower extract comprising Nymphae alba flower material; wherein the composition comprises from about 0.009% to about 0.015% of the Nymphae alba flower material and/or a hydrolysate thereof;
b. about 0.101% lactobionic acid;
c. a dermatologically acceptable carrier.

16. (canceled)

17. A skin care composition comprising:
a. about 0.4% Nymphae alba flower extract wherein the composition comprises from about 0.009% to about 0.015% of the water lily plant material and/or a hydrolysate thereof;
b. about 0.101% lactobionic acid;
c. a dermatologically acceptable carrier.

18. The skin care composition of claim 17, further comprising about 5% niacinamide.

19. The skin care composition of claim 18, further comprising:
a. silicone oils;
b. glycerol;
c. disodium EDTA;
d. lauroyl lysine;
e. xanthan gum;
f. benzyl alcohol; and
g. phenoxyethanol.

20. The skin care composition of claim 19, wherein the composition inhibits % yellowness by greater than 53% and inhibits % glycation by greater than or equal to 35% according to the Advanced Glycation End Product Assay.
